(19) United States
(12) Patent Application Publication
(10) Pub. No.: US 2010/0063346 A1
(43) Pub. Date: Mar. 11, 2010

(54) MAGNETORHEOLOGICAL FLUIDS AND THERAPEUTIC USES THEREOF

(76) Inventors: William J. Murphy, Reno, NV (US); Lisbeth A. Welniak, Reno, NV (US); Cahit Evrensel, Reno, NV (US); Alan Fuchs, Reno, NV (US); Faramarz Gordaninejad, Reno, NV (US)

Correspondence Address: UNR/DRI Technology Transfer Office, UNR-DRI Technology Transfer Office, Mail stop 321, 1664 N. Virginia St, Reno, NV, 89557, US

(21) Appl. No.: 12/300,843

(22) PCT Filed: May 17, 2007
(86) PCT No.: PCT/US2007/069170

§ 371 Date: Jan. 30, 2009

Related U.S. Application Data
(60) Provisional application No. 60/801,463, filed on May 17, 2006.

(51) Int. Cl. A61N 2/00

(52) U.S. Cl. 600/12

(57) ABSTRACT

In some embodiments, the present disclosure provides methods for stimulating a subject's immune system. Particular methods provide a tumor specific immune response by injecting a tumor with a magnetorheological fluid and applying a magnetic field to the tumor. The immune response can be used to treat disseminated tumors or metastatic cells. Further methods induce a general immune response, such as by treating a section of tissue, such as healthy tissue, with a magnetorheological fluid and applying a magnetic field to the treated tissue. The immune response can be used to treat illness or disease or to enhance the effect of a therapeutic agent. In particular examples, the disclosed methods are used to enhance the effect of immuno stimulants. Also provided are methods for causing necrosis of a cell mass, such as a tumor, by injecting a magnetorheological fluid into a section of tissue and applying a magnetic field to the tissue.

### CROSS REFERENCE TO RELATED APPLICATIONS

This application claims the benefit of, and incorporates by reference, U.S. Provisional Patent Application No. 60/801,463, filed May 17, 2006.

### FIELD

The present disclosure relates to magnetorheological fluids and their therapeutic uses. In particular aspects, the present disclosure relates to the use of magnetorheological fluids in cancer therapy, such as in the treatment of neoplastic tissue, such as solid tumors.

### BACKGROUND

Current treatment regimens in cancer often involve surgery followed by systemic chemotherapy and/or radiation. However, these treatments typically only slow the cancer and offer partial regression of the disease. Such treatments may not be suitable for the treatment of advanced disease. In addition, present systemic cancer treatments typically reduce or eliminate a subject's natural anti-tumor immune response, such as by killing immune effector cells.

In addition, removal of a primary tumor can have some negative consequences. For example, removal of the primary tumor can remove the stimulus, such as antigen presentation, for the subject's immune system, reducing or eliminating the subject's immune response to the cancer. In addition, the primary tumor can sometimes metastasize during removal.

### SUMMARY

The present disclosure provides compositions and methods for therapeutic treatment of a subject using a magnetorheological fluid. In particular examples, the magnetorheological fluid is administered with a therapeutic agent, such as an immunostimulant.

In particular embodiments, the present disclosure provides methods for treating neoplastic tissue, such as tumors. In certain disclosed methods, an effective amount of a magnetorheological fluid is administered to an area of a subject having neoplastic tissue. A magnetic field is applied to the neoplastic tissue. When the magnetic field is applied, neoplastic cells in the neoplastic tissue undergo necrosis. In some methods, the neoplastic tissue is removed from the subject following magnetic treatment.

The present disclosure also provides methods for therapeutically treating a subject. A magnetorheological fluid is administered to an area of the subject. A magnetic field is applied to the area. Application of the magnetic field provides therapeutic treatment to the subject. In particular examples, the therapeutic treatment is achieved by activating the immune system of the subject, such as by generating pro-inflammatory cytokines.

In further embodiments, the present disclosure provides methods for treating a subject having metastatic disease. A magnetorheological fluid is injected into a primary tumor of the subject. A magnetic field is applied proximate the primary tumor. Applying the magnetic field proximate the primary tumor activates the subject's immune system to attack metastatic cells. In particular methods, the primary tumor is removed from the subject following magnetic treatment.

The present disclosure also provides compositions useable for immunostimulation or cancer treatment. The compositions include a magnetic agent. The magnetic agent is suspended in a carrier. The compositions further include a therapeutic agent, such as an immunostimulant. In particular examples, the immunostimulant is at least one of IL-2 and anti CD-40.

In various implementations of embodiments of the present disclosure, the magnetic agent and magnetorheological fluid have an average cross sectional width of between about 100 nm and about 10 μm, about 0.5 μm to about 8 μm, about 3 μm to about 7 μm, or about 4 μm to about 5 μm. Although the magnetic agent may be composed of any suitable material, in particular examples the magnetic agent is composed of iron particles, including iron compounds or iron alloys. In some examples, the magnetic agent is coated, such as with a polymer coating, to improve biocompatibility. In a specific example, the polymer is poly(N-isopropylacrylamide).

In particular implementations of methods where a magnetic field is applied to a subject, including a portion of a subject, the magnet may have a strength of at least about 0.2 T, such as between about 0.2 T and about 2 T, between about 0.2 and about 1 T, between about 0.2 T and about 0.6 T, or between about 0.4 T and about 0.6 T. In a specific example, the magnet has a strength of about 0.5 T. In these implementations, the magnetic field may be applied for at least about 0.5 seconds, such as between about 15 seconds and about 20 minutes, between about 30 seconds and about 15 minutes, or between about 1 minute and about 10 minutes. In a specific example, a 0.5 T magnet is held proximate a subject for about 10 minutes. The treatment may be repeated in these implementations, including on a daily basis, such as 1, 2, 3, 4, or 5 treatments per day for between 1 and 10 days, 3 and 7 days, or 4 and 5 days. In a specific example, the treatment is applied once per day for four days.

In at least certain disclosed methods, the application of the magnetic field causes the magnetorheological fluid to disrupt the vascular system of a tumor or causes necrosis of tumor tissue. In further embodiments, the application of a magnetic field to an area of a subject having a magnetorheological fluid activates the immune system of the subject, such as by increasing exposure of the subject's immune system to an antigen. In particular examples, the methods do not derive therapeutic effect from blocking tumor vasculature. In various implementations of the present disclosure, magnetorheological treatment is combined with treatment with a therapeutic agent, such as an immunostimulant, for example, at least one of anti CD-40 and IL-2.

In yet further embodiments, the present disclosure provides a kit including a magnet, a magnetorheological fluid, a therapeutic agent, such as an immunostimulant, and, optionally, a syringe.

There are additional features and advantages of the various embodiments of the present invention. They will become evident as this specification proceeds.

In this regard, it is to be understood that this is a brief summary of the various embodiments described herein. Any given embodiment of the present invention need not provide all features noted above, nor must it solve all problems or address all issues in the prior art noted above.

BRIEF DESCRIPTION OF THE DRAWINGS

Various embodiments are shown and described in connection with the following drawings in which:

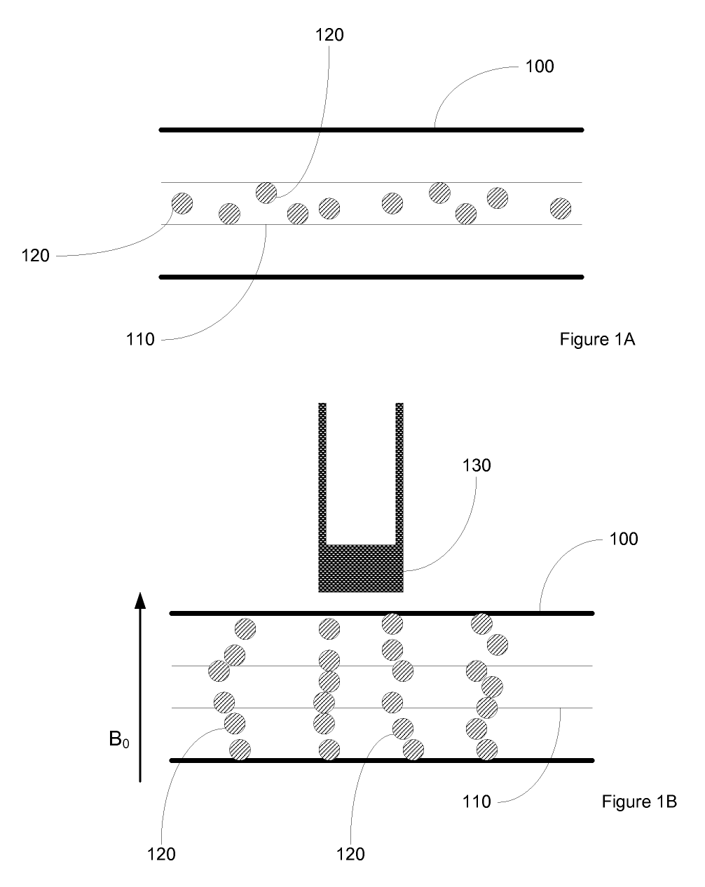

FIGS. 1A and 1B are schematic diagrams of a magnetorheological fluid in a tumor blood vessel before (FIG. 1A) and after (FIG. 1B) application of an external magnetic field.

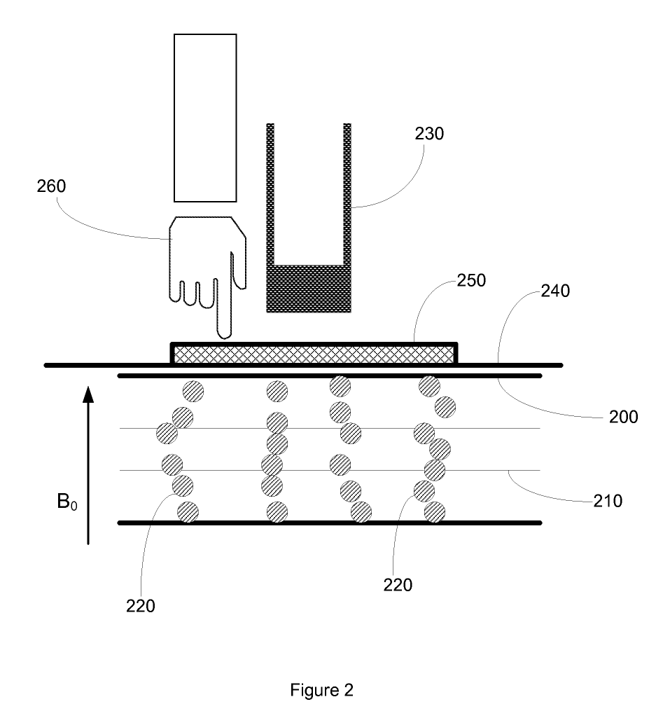

FIG. 2 is a schematic diagram of a swatch of material having a reinforced portion on the surface of a subject having a tumor.

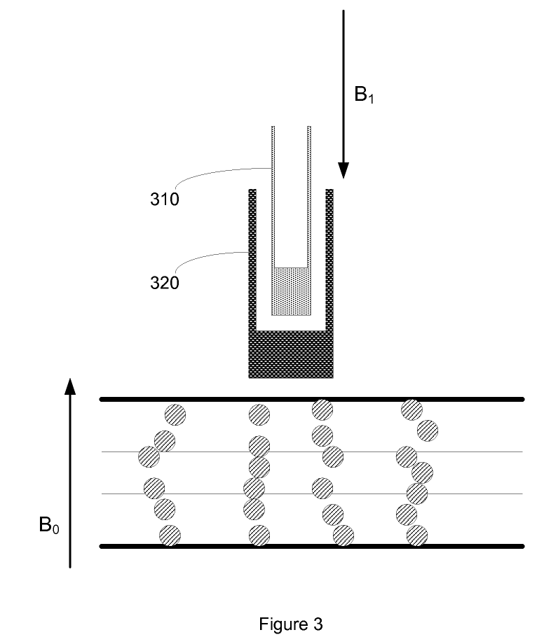

FIG. 3 is a schematic diagram of a second magnet being used to cancel at least a portion of a first magnet field to aid in removal of the first magnet from the surface of a subject.

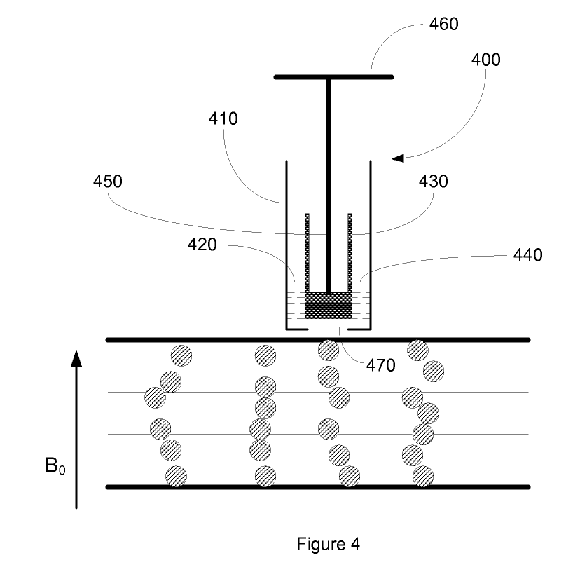

FIG. 4 is a schematic diagram of a screw-type device useable to remove a magnet from proximity to the surface of a subject.

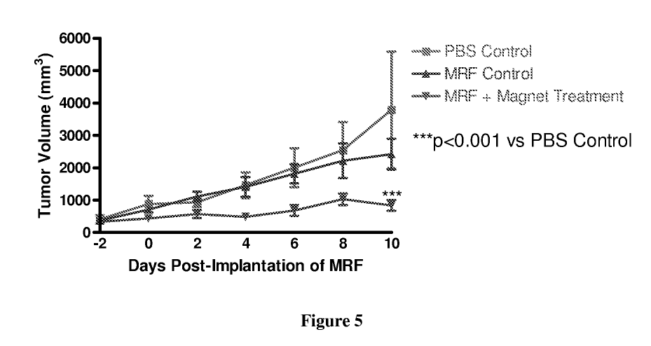

FIG. 5 is a graph of tumor volume versus days post-implantation of a magnetorheological fluid for three groups of mice treated with either phosphate buffered saline, magnetorheological fluid, or magnetorheological fluid with magnet treatment.

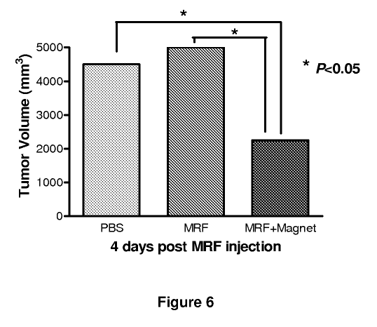

FIG. 6 is a graph of tumor volume after four days of treatment for three groups of mice treated with either phosphate buffered saline, magnetorheological fluid, or magnetorheological fluid with magnet treatment.

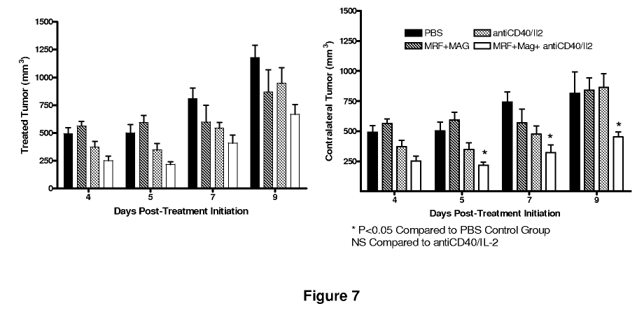

FIG. 7 is graphs of tumor volume versus days post-implantation of a magnetorheological fluid for treated and contralateral tumors in four groups of mice treated with either phosphate buffered saline, immunostimulants, magnetorheological fluid with magnet treatment, or magnetorheological fluid and immunostimulants with magnet treatment.

### DESCRIPTION

Unless otherwise explained, all technical and scientific terms used herein have the same meaning as commonly understood by one of ordinary skill in the art to which this disclosure belongs. In case of any such conflict, or a conflict between the present disclosure and any document referred to herein, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together. All numerical ranges given herein include all values, including end values (unless specifically excluded) and intermediate ranges.

Although methods and materials similar or equivalent to those described herein can be used in the practice or testing of the present disclosure, suitable methods and materials are described herein. The disclosed materials, methods, and examples are illustrative only and not intended to be limiting.

“Subject,” as used herein, refers to animals to be treated using the methods, compositions, and systems according to the present disclosure. Subjects include mammals, such as humans or veterinary animals, including a horse, a cow, a sheep, a pig, a dog, or a mouse.

As used herein, “magnetorheological fluid” or “MRF” is a fluid which is in a liquid or flowable state until a magnetic field is applied. When a magnetic field is applied, the fluid is converted to a semi-solid. Suitable MRFs include those using a magnetic agent, agents responsive to a magnetic field. Magnet agents include ferromagnetic, paramagnetic, or superparamagnetic substances.

Suitable magnetic agents include iron particles (“ferrous MRFs”) or iron compounds or alloys, including substances such as iron oxide, iron carbide, iron nitride, carbonyl iron, and steel materials, including silicon steel, stainless steel, and low carbon steel and alloys with one or more metals, such as copper, tungsten, silicon, chromium, manganese, aluminum, cobalt, vanadium, nickel, or molybdenum. For example, the MRF may be a fluid incorporating magnetite particles, such as those available from Chemicell GmbH (Germany). However, any suitable magnetic agent can be used, including ceramic particles, such as iron oxide materials. In particular implementations, the magnetic agent is selected to be biocompatible, or at least substantially biocompatible, such that the magnet agent is relatively inert after MRF treatment, such as being retained within a subject without eliciting any undesirable local or systemic effects.

The magnetic agent is suspended in a carrier, such as a biologically suitable carrier. Suitable carriers include water, buffered aqueous solutions, such as phosphate buffered saline solution, aqueous alcoholic mixtures, oils, and oil mixtures.

A magnetic agent may be chosen to have a suitable size for desired pharmacokinetic and therapeutic properties. For example, the size and shape of the magnetic agent may be chosen to have a particular effect upon the application of a magnetic field, such as to align in a particular manner or travel a particular distance. In particular embodiments, the particle size is chosen to facilitate clearance from the subject. In other embodiments, the particle size is chosen to facilitate retention of the particles in the treatment site.

The surface or shape of the particles may be chosen to provide desired physiological properties. For example, smooth, spherical particles may be better tolerated physiologically than sharp, angular particles. However, sharp, angular particles may be more effective in damaging an area to be treated, such for generating an immune response or causing cell necrosis. In particular examples, the particles of magnetic agent are at least substantially spherical.

Particular applications use particles having a diameter, or average cross sectional width, of between about 100 nm and about 10 μm, such as between about 0.5 μm and about 8 μm. In more particular examples, the particles have a size of between about 3 μm and about 7 μm, such as between about 4 μm and about 5 μm. In various implementations, the MRF can be composed of particles having a relatively uniform size or of particles having a plurality or range of sizes. Mixing particles of different sizes may be useful in controlling the response of the MRF to a magnetic field, tailor the effect of the MRF on tissue, or aid in administration or tolerance.

The MRF may include suitable agents to make it biocompatible. The MRF (and/or magnetic agent) may also be treated to aid in elimination of the MRF from the subject, retention of the MRF at the treatment site, or targeting of MRF particles, such as by attaching a tumor targeting substance to the particles.

In particular implementations, the magnetic agent is coated with a substance to enhance its biocompatibility. For example, magnetic agent particles may be coated with a polymer. In some examples, the polymer is a hydrophilic starch or starch derivative, gelatin, cellulose, or other naturally occurring polymer. In further examples, the polymer is a hydrogel, such as N-isopropylacrylamide (NIPAAm) polymers.

Additional polymers, and methods for coating magnetic agents with them, are disclosed at column 2, line 16 to line 52 and column 3, line 34-column 7, line 32 of U.S. Pat. No. 5,989,447, which portion is incorporated by reference herein. Additional coatings and techniques are disclosed in U.S. 2005/0109976, paragraphs 63-73 and 104-110 of which are hereby incorporated by reference. In some configurations, the coatings are applied to the magnetic agent particles by depositing the coating in liquid form, such as dissolved in a solvent, on the magnetic agent, such as through precipitation or evaporation. In further embodiments, the polymer is formed by reaction with a functional group on the surface of the magnetic agent by polycondensation, polyaddition, or polymerization of organic monomer units.

Surface initiated polymerization, also called grafting, may be used to coat the surfaces of micron particles with polymer materials for various functionalities. In this method, polymerization initiators are chemically bonded onto the surface of particles. Such surface modified particles behave as macroinitiators for ATRP. Well-defined polymer chains are grown from the particle surfaces to yield particles composed of inorganic particle core and polymer shell. Grafting can improve the stability of the polymer layer on the particle surface and increase the compatibility of the particles with the fluid system and monomer. This approach can provide flexibility in controlling the molecular structure of polymers.

After fabrication, the coated magnetic agent particles may be purified by any suitable means, including applying a small magnetic field to a sample and draining the excess solution. This process can be repeated several times to remove any non-polymeric fractions and any other materials that may have formed in the medium and not on the particle surface. The particles can also be purified by dialysis, such as using de-ionized water and Spectra/Por Biotech Regenerated Cellulose tubular dialysis membranes with a molecular weight cutoff of 15,000 Daltons. In particular examples, the particles may be further purified using a Cellu Sep Regenerated Cellulose Tubular Dialysis Membrane with a molecular weight cutoff of 6,000-8,000 Daltons.

The MRF may include additional agents. For example, the MRF may incorporate an imaging agent so that the area to be treated can be imaged, such as by magnetic resonance imaging or x-ray. Imaging the area may aid in applying the magnetic field to a subject. The MRF may include a targeting agent, such as an agent with an affinity for neoplastic cells. The MRF may also include additional therapeutic substances, including additional antitumor agents or immunostimulants.

The MRF may be administered in any appropriate manner. For example, the MRF may be administered by intravenous injection, parenteral injection, peritoneal injection, subcutaneous injection, intracutaneous injection, intratumoral injection, peritumoral injection, injection into the lymphatic system, injection into a surgical field, or subdermal injection. Other means of administration can be used, including oral, buccal, sublingual, and rectal administration and by intravenous or intraperitoneal infusion. In particular embodiments, the MRF is injected intratumorally. Without being bound by theory, the MRF may then diffuse into the tumor vasculature.

The MRF can be administered in any suitable concentration. In various embodiments, the MRF has a particle concentration of about 40% to about 85% wt/vol of MRF solution, such as about 50% to about 85% or about 65% to about 85% wt/vol. In particular examples, the MRF has a particle concentration of about 80% wt/vol of MRF solution. In particular configurations, the MRF is administered as a phosphate buffered saline (PBS) solution, such as an 80% w/v solution of MRF/PBS.

In particular aspects, the MRF is injected in an effective amount. An effective amount is an amount of MRF (or magnetic agent) sufficient to inhibit the growth, and/or cause necrosis of, neoplastic tissue. In further aspects, the MRF is injected in an amount effective to induce an immune response in a subject, such as increasing exposure of a subject's immune system to an antigen. The amount of MRF used in a particular method may depend on the size or volume of the subject to be treated. In some examples, when an 80% wt/vol suspension of MRF, such as an MRF containing 3-5 micron iron particles, is used, the MRF is administered at a rate of about 10 μl for between about 0.25 mm³ and about 50 mm³, about 1.0 mm³ to about 10 mm³, or about 1.5 mm³ to about 5 mm³ of treatment area. The volume of MRF needed for a particular application may also depend on other factors, such as the nature of the MRF, the location of the area to be treated, the magnet to be used in the treatment, and the particular treatment regime to be used.

The treatment area, such as a tumor, may be any suitable size to cause a desired degree of tissue necrosis or achieve a desired therapeutic response. In particular examples, the treatment area has a volume of between about 1 mm³ and about 1000 cm³, about 50 mm³ and about 500 cm³, about 100 mm³ and about 400 cm³, or about 150 mm³ and about 300 cm³.

“Magnet” refers to devices and substances for generating a magnetic field. Magnetic field can be measured in Tesla. In particular examples, the magnet has a strength of about 0.2 T to about 2.0 T, such as between about 0.2 T to about 1 T. In further examples, the magnet has a strength of at least about 0.2 T, such as a strength between about 0.2 T and about 0.6 T or about 0.4 T and about 0.6 T. In a specific example, the magnet has a strength of about 0.5 T. The strength of the magnet used in a particular technique can be varied based on a number of factors, including the nature of the MRF, including the composition of the particles in the MRF and their size, the size of the tumor, the location of the tumor, the size or weight of the subject, and the distance between the magnet and the MRF after introduction of the MRF into the subject. For example, stronger magnets may be needed with a MRF having smaller particles, as larger particles can exhibit a stronger magnetic response. In some examples, the magnet is an electromagnet and can be selectively switched on and off.

When the present methods are used for applications close to the surface of a subject's skin, such as for treating primary tumors located close to the surface of the skin, the magnet is placed in contact with the subject's skin in certain implementations. In further implementations, the magnet is located within an effective distance, such as less than a few inches, from the area to be treated. When the area to be treated is located within a subject, the magnet may be placed proximate the area through any suitable means, such as insertion through a body orifice or cavity, or using surgical techniques.

FIG. 1A illustrates a surface tumor 100 having a blood vessel 110, such as a vein or capillary. A plurality of magnetic agent particles 120 located in the blood vessel 110. No external magnetic field has been applied to the tumor 100 and so the particles 120 are randomly oriented.

FIG. 1B illustrates the tumor 100 after an external magnetic field B₀ has been applied by placing a magnet 130 proximate the tumor 100. The particles 120 have aligned into chain-like structures. In forming chains, some of the particles 120 have been pulled outside of the blood vessel 110, disrupting the vasculature and perforating the tumor 100.

In some examples, a material, such as fabric, is placed between the magnet and the subject's skin. The particular configurations, the material may be wrapped around a portion of the subject. In more specific examples, the material has a reinforced portion which can be used for leverage when removing the magnet. FIG. 2 illustrates a surface tumor 200 having a blood vessel 210, such as a vein or capillary. A plurality of magnetic agent particles 220 have formed into chains due to a magnet field B₀ applied using a magnet 230. A swatch of material 240 is placed over the tumor 200, between the surface of the tumor 200 and the magnet 230. The material 240 has a thicker, reinforced section 250. The material 240 and section 250 can aid in removal of the magnet from the subject by providing a place for a user's hand or fingers 260 to exert leverage while pulling on the magnet 230.

Other techniques can be used to aid in removing a magnet from a subject. For example, a second magnet may be placed on or proximate the treating magnet, or the subject, in order to reduce or cancel the magnetic field created by the treating magnet. This embodiment is illustrated in FIG. 3, where a second magnet 310 having a magnetic field B₁ is placed proximate a first magnet 320 having a field B₀ oriented in the opposite direction of B₁. In specific examples, the second magnet 310 is an electromagnet.

In yet further embodiments, the magnet can include mechanical structures to aid in its removal from proximity to a subject. For example, FIG. 4 illustrates a device 400 having a cylindrical housing 410 having threads 420 at a portion proximate a subject. A magnet 430 includes threads 440 which are matingly received by the threads 420. The magnet 430 includes a shaft 450 and a handle 460. The magnet 430 can be moved within the housing 410 by twisting the handle 460. The bottom of the housing 410 has a contact section 470 which may be placed against the surface of a subject. The contact section 470 is made of a material that sufficiently transmits the magnetic field from the magnet 430 and is thick enough to allow such transmission. In particular examples, the contact section 470 is made of stainless steel or fabric. The contact section 470 is omitted in some embodiments, such as a device 400 with a housing 410 which extends under the magnet 430.

The magnet is typically placed proximate the subject for a time sufficient to cause the particles in the MRF to respond to the magnetic field, such as aligning within, and perforating, a desired section of tissue. The time the magnetic field is applied may vary based on a number of factors, such as the size of the MRF particles, the nature of the MRF, the strength of the magnet, the distance between the subject and the magnet, and the depth within the subject of the MRF or tissue section. In various examples, the magnetic field is applied for at least about 0.5 seconds, such as about 15 seconds to about 20 minutes, about 30 seconds to about 15 minutes, or about 1 minute to about 10 minutes.

In some embodiments, the MRF treatment is applied a single time. In further embodiments, the MRF treatment is applied multiple times, such according to a schedule, such as 1, 2, 3, 4, or 5 treatments per day for between 1 and 10 days, 3 and 7 days, or 4 and 5 days. In particular examples, the treatment is applied 2 to 5 times, such as 2 to 3 times. When multiple treatments are used, successive treatments may be staggered, such as waiting 2, 3, 4, 6, 8, 12, 18, 24, 36, or 48 hours between treatments. In some methods, additional MRF is administered to the subject prior to successive exposure to a magnetic field. In further methods, the subject is exposed multiple times to a magnetic field without administering additional MRF.

“Therapeutic agent” refers to compounds, substances, or compositions having anti-cancer activity. Certain therapeutic agents have anti-tumor activity when in the vicinity of tumor cells. Other therapeutic agents are taken up by tumor cells, or have a greater therapeutic effect when taken up by tumor cells. Yet further therapeutic agents have an indirect effect on cancer cells, such as by stimulating a subject's immune system which then attacks cancer cells.

Examples of anti-tumor agents include antimetabolites, alkylating agents, hormones and their antagonists, natural products, or immunostimulants. Examples of alkylating agents include alkyl sulfonates (such as busulfan), nitrogen mustards (such as uracil mustard, chlorambucil, mechlorethamine, cyclophosphamide, or melphalan), nitrosoureas (such as carmustine, streptozocin, lomustine, semustine, or dacarbazine). Examples of antimetabolites include pyrimidine analogs (such as 5-FU or cytarabine), folic acid analogs (such as methotrexate), and purine analogs, such as thioguaninemer or captopurine. Examples of natural products include antibiotics (such as doxorubicin, bleomycin, plicamycin, dactinomycin, daunorubicin, or mitocycin C), vinca alkaloids (such as vincristine, vinblastine, or vindesine), epipodophyllotoxins (such as teniposide or etoposide), and enzymes (such as L-asparaginase). Examples of miscellaneous agents include platinum coordination complexes (such as cis-diamine-dichloroplatinum II also known as cisplatin), methyl hydrazine derivatives (such as procarbazine), substituted ureas (such as hydroxyurea), and adrenocrotical suppressants (such as aminoglutethimide and mitotane). Examples of hormones and antagonists include estrogens (such as diethylstilbestrol and ethinyl estradiol), antiestrogens (such as tamoxifen), adrenocorticosteroids (such as prednisone), progestins (such as hydroxyprogesterone caproate, medroxyprogesterone acdtate, and magestrol acetate), and androgens (such as testerone proprionate and fluoxymesterone). In particular examples, the therapeutic substances includes one or more of Fludarabine, Navelbine, Adriamycin, Velban, DTIC, Alkeran, Mitomycin, Ara-C, BiCNU, Busulfan, CCNU, Carboplatinum, Cisplatinum, Herceptin, Xeloda (Capecitabine), Cytoxan, Daunorubicin, 5-FU, Irinotecan (Camptosar, CPT-11), Hydrea, Nitrogen Mustard, Idarubicin, Taxotere, Ifosfamide, Methotrexate, Zevelin, Mithramycin, Rituxan, STI-571, Mitoxantrone, Taxol, Vincristine, VP-16, Gemcitabine (Gemzar), Leustatin, Topotecan (Hycamtin), and calcitriol.

“Immunostimulation,” as used herein, refers to the stimulation of the immune system of a subject, either inducing activation or increasing activity, such as through administration of a chemical, composition, or biological substance, or through physical means, all collectively referred to as “immunostimulants.” Immunostimulants include vaccines, adjuvants, antigens, and signaling compounds such as isoprinosine, and cytokines, such as interleukins and interferons, such as interferon alpha. Specific examples of immunostimulants include loxoribine, bacterial DNA, and lipopolysaccharide. Adjuvants which can be used as immunostimulants include aluminum hydroxide; aluminum phosphate; calcium salts; iron salts; zinc salts; mineral oil; Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); Cytokines, such as GM-CSF, interleukin-2, -7, -12, and other like growth factors, cationically or anionically derivatized polysaccharides; acylated tyrosine; acylated sugars; polyphosphazenes; biodegradable microspheres; monophosphoryl lipid A, such as 3-de-O-acylated monophosphoryl lipid A, and saponin, such as Quil A, including derivatives such as QS21 and QS7, Escin, Digitonin, Gypsophila, Chenopodium quinoa saponins; Montanide ISA 720 (Seppic, France); SAF (Chiron, Calif.); ISCOMS (CSL); MF-59 (Chiron); the SBAS adjuvants (such as SBAS-2 or SBAS-4, available from SmithKline Beecham, Belgium); Detox (Enhanzyn RTM) (Corixa, Hamilton, Mont.); RC-529 (Corixa) and other aminoalkyl glucosaminide 4-phosphates (AGPs); and polyoxyethylene ether adjuvants.

For example, the immune system of a subject can be stimulated through CD40 stimulation, such through administration of anti-CD40. The immune system can also be stimulated through the administration of cytokines, such as IL-2. Additional immunostimulant techniques can be used in the methods of the present disclosure, including combinations of techniques, such as administration of IL-2 and anti-CD40.

Treatment of Primary Tumor

Methods of the present disclosure can be used to treat primary tumors. A MRF is administered to a subject such that the MRF concentrates in the vicinity of the tumor, such as in the tumor vasculature. In particular examples, the MRF is injected into the tumor.

After the MRF has concentrated in the tumor, and/or tumor vasculature, to a desired degree, a magnetic field is applied to the affected area. As the MRF solidifies (or otherwise increases in particle size or density), the tumor vasculature is disrupted, in certain embodiments. For example, the vasculature may be sheared or otherwise rendered non-functional. The neoplastic cells, and normal cells within the treated area, fed by affected vasculature then undergo necrosis. In particular embodiments, the anti-neoplastic effect of the MRF is not due to the formation of a seal which blocks the flow of blood through the tumor vasculature.

After the MRF treatment, the tumor may be removed using conventional means. For example, the tumor may be surgically removed after a desired amount of MRF treatment.

Tumor removal after MRF treatment may provide a number of advantages. For example, MRF can result in necrosis of the tumor tissue, which can help prevent the tumor from spreading during removal. MRF treatment can also allow the growth of a tumor to be reduced or arrested until tumor removal can be accomplished. In addition, leaving the necrotic cancer tissue in the subject may aid the subject's immune system to mount a response to the cancer.

Immune System Activation and Disseminated Treatment

In further embodiments, the present disclosure provides methods for activating the immune system of a subject. According to the method, a subject is treated with a MRF. The subject, or a portion thereof, is subjected to a magnetic field. The reaction of the MRF to the applied magnetic field generates an immune response in the subject, such as immune cell recruitment and activation.

In particular implementations, the immune system of a subject can be activated to attack neoplastic cells, such as by exposing a subject's immune system to a greater amount of an antigen. For example, when a primary tumor is treated with a MRF as described above, the neoplastic cells or normal cells may emit signals, such as “danger signals,” as they undergo necrosis. Without being bound by theory, these signals can involve the engagement of Toll-like receptors and the production of pro-inflammatory cytokines. The generation of such signals may be important in the generation of an effective immune response. These signals may promote an anti-tumor response in the immune system of the treated subject. Compared to other method of treating neoplastic tissue, since no cytoreductive therapies are being given, the immune cells are unhindered and may attack tumors.

The enhanced immune response is used, in some methods, to treat disseminated tumors or neoplastic cells, such as secondary tumors or metastatic cells. A subject with metastatic cancer may be treated by injecting an MRF into a primary tumor and subjecting the tumor to a magnetic field. These methods can be particularly advantageous because they do not require disseminated tumors to be located in order to be treated. The subject's immune system will seek out and attack cancerous cells. Thus the present disclosure provides a cancer vaccine which lacks disadvantages of conventional techniques, such as removal and re-injection of a tumor in order to generate an immune response.

In some methods a tumor or other tissue is treated with MRF and a magnetic field to achieve a therapeutic effect, such as an immune response specific for a particular disease or illness. In further methods, normal tissue is treated with MRF and a magnetic field to achieve a therapeutic effect, such as a general immune response. Inducement of a general immune response can be used to treat various diseases or illnesses, including neoplastic cells, or to enhance other treatments or therapeutic agents. For example, MRF treatment can enhance the activity of immunostimulants such as CD-40 and IL-2. MRF can also be used to enhance a subject's immune response to a vaccine, such as a cancer, flu, tuberculosis, or anthrax vaccine.

Compositions

The present disclosure provides compositions for treating cancer or immunostimulation. The compositions include a magnetorheological fluid, which includes a magnetic agent and a carrier. The compositions further include a therapeutic agent, such as an immunostimulant. In particular examples, the compositions include one or both of IL-2 and anti CD-40.

The present disclosure also provides kits for treating cancer or immunostimulation. The kit includes a magnet, a magnetorheological fluid, and a therapeutic substance, such as an immunostimulant. In particular examples, the kit includes one or both of IL-2 and anti CD-40. Kits can include additional substances or devices, including patches to place between the skin of a subject and a magnet, other tools to facilitate magnet removal, or a syringe for administering MRF or therapeutic agent to a subject.

The present disclosure thus provides methods, compositions, and systems to disrupt primary tumors, provide direct anti-tumor effects, and augment a subject's immune response. Compared with prior methods, the present disclosure can provide a number of advantages. For example, the magnetorheological fluids can be relatively non-toxic and, at least in some examples, are retained at the treatment site even after multiple applications of a magnetic field. The methods also may be beneficial because the treatment is not toxic to normal cells and thus may avoid the side effects of conventional treatments such as chemotherapy and radiation therapy.

The present disclosure can be used to enhance the effect of other therapeutic agents, including immunostimulants. For example, the disclosed methods can be used to enhance the effect even of the potent, synergistic combination of anti-CD40 and IL-2, which are currently undergoing clinical trials for cancer treatment. Compared to other methods of immunostimulation, the presently disclosed methods can be beneficial as tissue damage can be controlled, including the extent and duration of injury used to generate a desired immune response.

### EXAMPLE 1

### MRF Treatment of Primary Tumors in Mice

Mice were given the breast carcinoma lines 4T1 (FIG. 5) or B16 (FIG. 6), subcutaneously. After a period of time in which palpable tumors were obtained (an average volume of 370 mm³ for 4T1 and 380 mm³ for B16), the mice were divided into three experimental groups.

A magnetorheological fluid (100 μl) composed of 3-5 micron iron particles suspended in phosphate buffered saline solution (PBS; 80% w/v) was injected into subcutaneous (s.c.) B16 melanoma tumors in C57BL/6 mice. One group of mice received PBS (carrier fluid) alone. A second group received no further treatment following the administration of the MRF. A third group was treated by placing a 0.4 Tesla strength magnet over the tumor for 10 minutes following the injection of the MRF and then daily for a total of 4 (FIG. 6) or 5 treatments (FIG. 5).

The treatment was well tolerated. No toxicity was observed in measurements of kidney or liver function. The maintenance of MRF at the site of injection was discernible by the persistence of magnetic attraction to the MRF in the tumor during a 7 day period. Significant inhibition of tumor growth was observed in the treated mice compared to mice that received PBS or MRF without magnetic field treatment (FIGS. 5 and 6). The tumors of the treated mice displayed a disrupted histological architecture suggesting that mechanical stress affected the tumor.

### EXAMPLE 2

### Treatment of Disseminated Tumors in Mice Using MRF and Immunostimulants

CD40 and its ligand, CD40L (CD154), are members of the TNF receptor and TNF ligand superfamilies, respectively. CD40 is present on a variety of cells including: B cells, monocytes, dendritic cells (DC), and endothelial cells. CD40 is also expressed on various neoplastic cells, including B cell lymphomas, breast carcinomas, bladder carcinomas, melanomas, ovarian carcinomas, and renal carcinomas. CD40L is present on activated T cells, NK cells, and platelets. This Example demonstrates that MRF and magnet treatment augments anti-tumor responses in combination with anti-CD40 and IL-2 treatment.

4TI breast cancer cells were implanted subcutaneously in the right and left flanks of BALB/c mice. When tumors reached an average volume of 175 mm³ the mice were treated with phosphate buffered saline, a suboptimal dose of anti-CD40 (65 μg) and IL-2 (50,000 IU) (administered by intra-peritoneal injection), a magnetorheological fluid (80% w/v MRF (3-5 μm iron particles) in 100 μl PBS), or magnetorheological fluid, anti-CD40, and IL-2. The use of PBS only, immunostimulant only, and MRF-only groups allowed any augmentation of response with MRF and magnet treatment or immunostimulant to be measured.

The following day magnet treatment was initiated in the ispolateral tumor. Groups receiving magnetic therapy were treated by placing a 0.4 T magnet over the tumor for 10 minutes a day for four consecutive days.

Suppression of tumor growth was observed in both the treated and the contralateral tumor in mice that received the combination of anti-CD40/IL2 immunotherapy and MRF/magnet treatment (FIG. 7) demonstrating the enhancement of systemic immune responses with this treatment. This tumor suppression was greater than tumor suppression observed in mice receiving only immunostimulant treatment. No enhancement in suppression was observed for MRF alone or immunostimulation alone after 9 days post-treatment initiation. This data suggests that metastatic tumors may be targeted for immunotherapy by a combination of systemic and localized treatment of a primary tumor.

### EXAMPLE 3

### Preparation of NIPAAm Coated Iron Particles

A polymerization reactor was assembled from components purchased from Ace Glass. The reactor consists of 1 L round bottom flask fitted with a 5 neck reaction flask head. The round bottom flask was placed in a heating mantle and a stirring motor capable of stirring a 10 L solution was situated above the reaction flask head. A blade stirrer was inserted through one of the necks of the reaction flask head. A thermocouple was inserted into one of the necks of the reaction flask head and placed in communication with a temperature controller and the heating mantle. Addition necks of the reaction flask head were used for a nitrogen line, condenser, and chemical addition. Reactions were performed under nitrogen.

A 1 mL 0.04 M aqueous initiator solution composed of de-ionized water and 3-chloropropionic acid was prepared. It was added to a 400 mL mixture of de-ionized water and 10 g 5 μm carbonyl iron particles. This mixture was stirred 12 h at 70° C. The particles were then filtered and washed several times to remove excess initiator. The particles were re-dispersed in 300 mL de-ionized water. A 1 mL 0.04 M CuBr, 0.1 M 4,4′-Bipyridine, and 2 M N-isopropylacrylamide (NIPAAm) aqueous solution was prepared and added to the surface initiated mixture. This mixture was stirred for 4 h at 70° C. The particles were filtered and washed then re-dispersed in a small amount of de-ionized water.

The coated particles exhibited a settling rate lower than the sample without the coating. The sample with highest ratio of polymer appeared to absorb a great deal of water, which may be due to the hydrophilic nature of the coating. The samples re-dispersed easily upon shaking. The magnetic properties of the coated particles were confirmed by applying a magnetic field to the sample.

The sample was tested by injection through a 22 gauge needle syringe. It was shaken before transfer to the syringe in order to improve dispersion. It flowed through the needle without plugging.

It is to be understood that the foregoing is a detailed description of certain embodiments. The scope of the present invention is not to be limited thereby and is to be measured by the claims, which shall embrace appropriate equivalents.

1. A method of treating neoplastic tissue comprising:
administering an effective amount of a magnetorheological fluid to an area of a subject comprising neoplastic tissue; and
applying a magnetic field to the area, whereby neoplastic cells of the neoplastic tissue undergo necrosis when the magnetic field is applied.

2. The method of claim 1, wherein the magnetorheological fluid comprises ferrous particles.

3. The method of claim 1, wherein the magnetorheological fluid comprises magnetite particles.

4. The method of claim 1, wherein the magnetorheological fluid comprises particles have a diameter of about 0.2 μm to about 10 μm.

5. The method of claim 1, wherein administering an effective amount of a magnetorheological fluid comprises injecting the magnetorheological fluid into a tumor of the subject.

6. The method of claim 1, wherein the magnetorheological fluid disrupts tumor vasculature when the magnetic field is applied.

7. The method of claim 1, wherein the magnetorheological fluid at least substantially does not derive anti-tumor effect from blocking tumor vasculature when the magnetic field is applied.

8. The method of claim 1, wherein applying a magnetic field to an area of a subject having neoplastic tissue comprises periodically applying a magnetic field to the neoplastic tissue over a time period.

9. The method of claim 1, wherein applying a magnetic field to an area of a subject having neoplastic tissue comprises applying the magnetic field for between about 1 and about 30 minutes.

10. The method of claim 1, wherein the applied magnetic field is between about 0.2 and about 1.0 Tesla.

11. A method of therapeutically treating a subject comprising:
administering a magnetorheological fluid to an area of a subject; and
applying a magnetic field to the area;
whereby applying the magnetic field to the area provides therapeutic treatment to a subject.

12. The method of claim 11, wherein the therapeutic treatment comprising activation of the subject's immune system.

13. The method of claim 12, wherein activating the immune system of the subject comprises increasing exposure of the subject's immune system to an antigen.

14. A method of treating a subject having metastatic disease comprising:
injecting a magnetorheological fluid into a primary tumor of a subject; and
applying a magnetic field proximate the primary tumor;
whereby applying the magnetic field proximate the primary tumor activates the subject's immune system to attack metastatic cancer cells.

15. The method of claim 14, further comprising administering an immunostimulant to the subject.

16. The method of claim 15, wherein the immunostimulant comprises antiCD40 and IL-2.

17. The method of claim 14, further comprising removing the primary tumor from the subject.

18. The method of claim 14, wherein the magnetorheological fluid comprises iron particles having an average cross section of about 3 to about 5 microns.

19-24. (canceled)
